(19) United States
(12) Patent Application Publication
(10) Pub. No.: US 2010/0010418 A1
(43) Pub. Date: Jan. 14, 2010

(54) ELECTRICALLY ACTIVATED GEL ARRAY FOR TRANSDERMAL DRUG DELIVERY

(75) Inventor: Giovanni Nisato, Eindhoven (NL)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N. V., Eindhoven (NL)

(21) Appl. No.: 12/442,959

(22) PCT Filed: Sep. 26, 2007
(86) PCT No.: PCT/IB2007/053918

§ 371 Date: Mar. 26, 2009

Related U.S. Application Data
(60) Provisional application No. 60/827,507, filed on Sep. 29, 2006.

(51) Int. Cl. A61N 1/30; A61M 35/00; A61K 9/70

(52) U.S. Cl. 604/20; 604/304

(57) ABSTRACT

A transdermal drug delivery system (100) for providing controlled doses of a drug through the epidermis of a human or other animal is disclosed. In one embodiment, the transdermal drug delivery system (100) includes a substrate (110) having an array of one or more electrode pairs (140) disposed thereon and a gel (130) disposed on the substrate (110) and in electrical contact with each electrode (142, 144) of the one or more electrode pairs (140). The gel (130) contains at least a first medicating agent and is configured to change a rate of release of the first medicating agent based on at least one of a voltage or current provided by the electrode pairs (140).

Broadly defined, a transdermal drug delivery system is any system designed to administer an appreciable dose of some drug directly through the skin without use of a conventional hypodermic needle. Examples of transdermal drug delivery systems include “the patch” (i.e., an adhesive patch design to deliver nicotine to tobacco-addicted people), aspirin-laced balms and adhesive patches designed to administer highly potent pain-killers.

While the hypodermic or oral administration of a drug is often the preferred method of drug delivery, transdermal drug delivery provides a number of advantages including the release of medication over prolonged periods and favorable patient feedback.

Unfortunately, existing transdermal patches lack versatility, and their effectiveness can be hampered by the kinetics of the drug used, skin interaction and drug solubility. Further, existing transdermal patches cannot provide periodic dosing or dosing on demand. Still further, there are a wide variety of drugs that are not readily absorbed by human skin. Accordingly, new technology related to transdermal drug delivery systems is desirable.

A transdermal drug delivery system is disclosed for providing controlled doses of a drug through the epidermis of a human or other animal. The transdermal drug delivery system includes a substrate having an array of one or more electrode pairs and a gel disposed thereon, wherein the gel is disposed in electrical contact with each electrode of the one or more electrode pairs, and wherein the gel contains at least a first medicating agent.

The various advantages offered by the disclosed methods and systems include providing a “drug on demand” system where, as opposed to conventional transdermal patches, doses can be delivered according to any number of predetermined schedules. Further, total dosage can be adjusted on the fly and adjusted from one patient to another taking into account different body weights or metabolisms.

The following detailed description is best understood when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

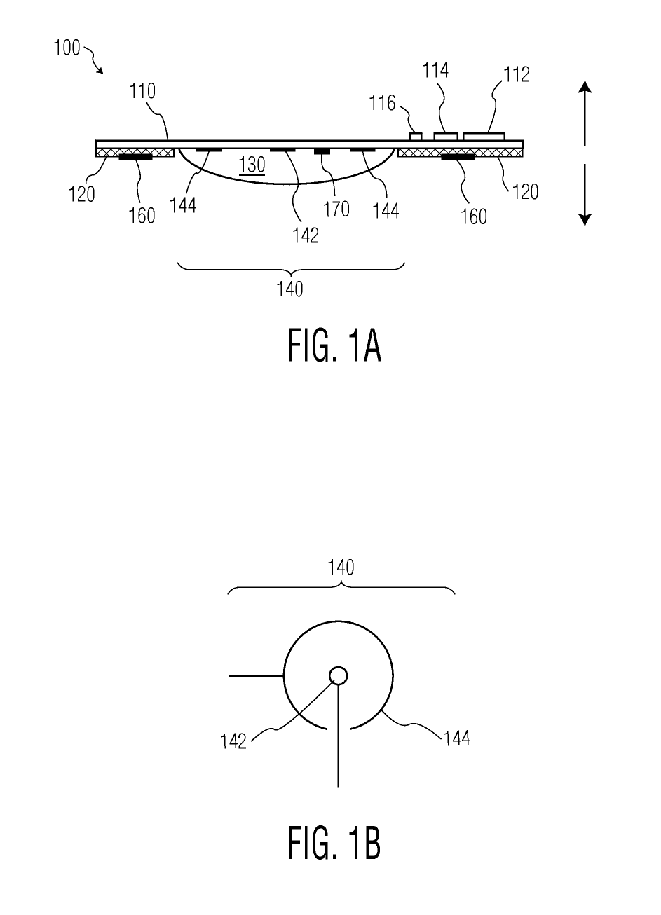

FIG. 1A is a cross-sectional side-view of an exemplary transdermal drug delivery system;

FIG. 1B depicts a top-down view of the exemplary electrode pair;

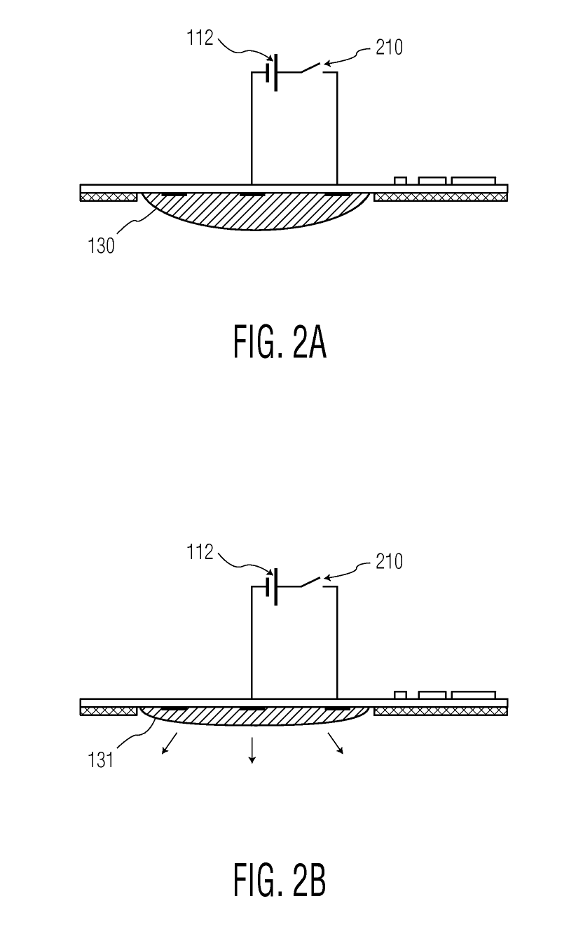

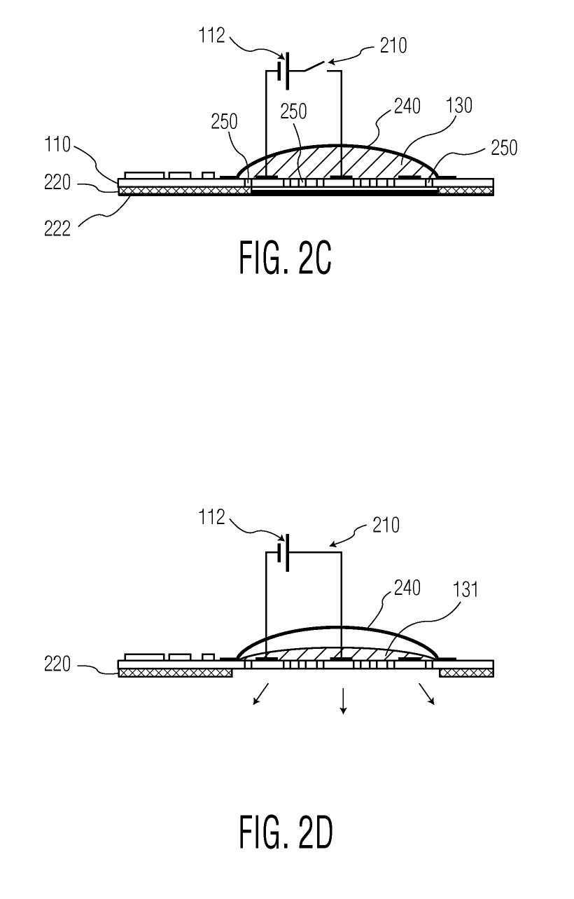

FIGS. 2A and 2B depict an exemplary process wherein a drug and solvent are controllably expelled from a gel in response to the application of an electric field using the delivery system of FIG. 1A;

FIGS. 2C and 2D depict a second exemplary process wherein a drug and solvent are controllably expelled from a gel in response to the application of an electric field using a variant of the delivery system of FIG. 1A;

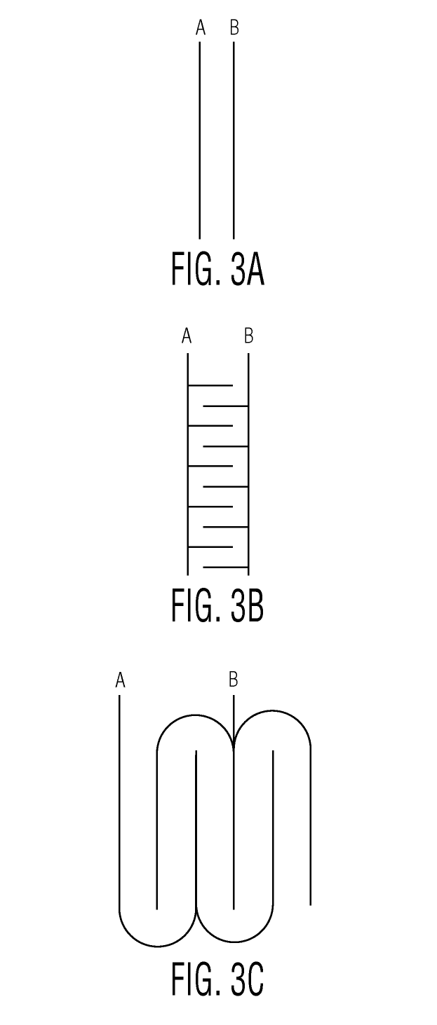

FIG. 3A-3C depict various exemplary electrode pairs for use with the disclosed methods and systems;

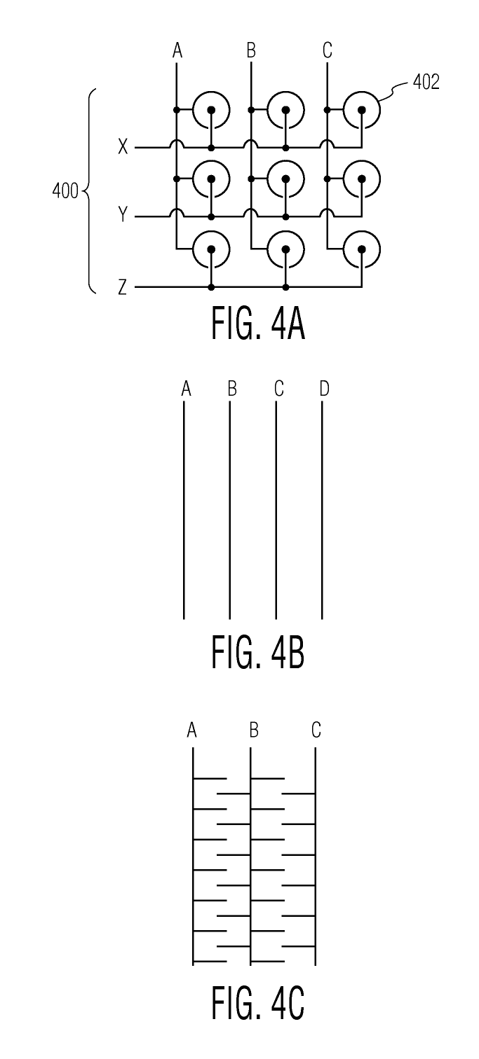

FIG. 4A-4C depict various exemplary electrode pair arrays for use with the disclosed methods and systems; and

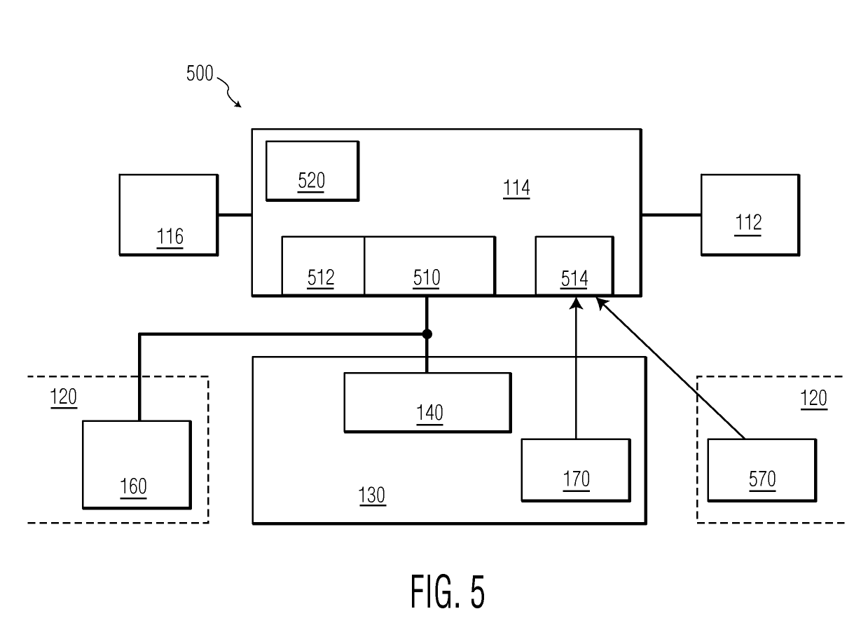

FIG. 5 is a block diagram of an exemplary transdermal drug delivery system.

In the following detailed description, for purposes of explanation and not limitation, specific details of exemplary embodiments are set forth in order to provide a thorough understanding of the present teachings. However, it will be apparent to one having ordinary skill in the art having had the benefit of the present disclosure that other embodiments according to the present teachings that depart from the specific details disclosed herein remain within the scope of the appended claims. Moreover, descriptions of well-known apparatus and methods may be omitted so as to not obscure the description of the exemplary embodiments. Such methods and apparatus are clearly within the scope of the present teachings.

FIG. 1A shows a transdermal drug delivery system 100 which includes a substrate 110 having a user interface 116, a controller 114 and a battery 112 disposed (directly or indirectly) upon the upper side of the substrate 110, and an electrode pair including electrodes 142 and 144 disposed (directly or indirectly) upon the lower side of the substrate 110. A gel 130 containing a solvent and one or more medicating agents, e.g., drugs, hormones, vitamins etc, is disposed on the bottom of the substrate in a fashion such that the gel 130 is in direct contact with the electrode pair. An optional sensor 170 is disposed within the gel 130. An optional barrier adhesive 120 is placed to surround the gel 130 to limit environmental exposure, to provide better adhesion to a person's skin and/or to provide a location to embed various sensors or other devices that may aid in transdermal drug delivery. Optional electrodes 160 are placed in the optional barrier adhesive 120.

The substrate 110 of FIG. 1A can be made from any number of metallic and non-metallic foils or fabrics, ceramic materials, plastic or cloth foils, or a composite thereof. Examples of plastics can include polymides, polynorbonene, polycarbonates, polyethersulfone and poly-ethylene therepthalate. The substrate 110 can additionally be provided with a water diffusion barrier layer to prevent desiccation of the gel. In various embodiments, such a diffusion barrier (not shown) can be made of thin metal layers, such as aluminum or aluminum oxides, silicon oxides, silicon oxinitrides and multiple layers thereof.

FIG. 1B shows a top-down view of the electrode pair of FIG. 1A. The electrode pair includes two electrodes 142 and 144 with the outer electrode 144 ranging in diameter from 50 microns to about 5 millimeters. The overall configuration of the electrode pair is shaped to produce an optimized (even??) electric field between the electrodes 142 and 144, but of course it may be appreciated that other electrode configurations can alternatively be used from embodiment to embodiment.

Electrodes 142 and 144 can take many forms including the form of various metal foils. Illustrative metal foils include, but are not limited to: copper, silver or gold, platinum, molybdenum and chromium (and multilayer combinations thereof). The foils can also take the form of a conductive ink or other conductive medium that may be deposited on or in the substrate 110. The foils are provided on or over the substrate 110. However, it should be appreciated that the particular makeup of a given pair of electrodes can vary from embodiment to embodiment as may be found necessary or advantageous.

The gel 130 of FIG. 1A is a polyelectrolyte substance configured to expel a solvent, such as water, in the presence of an electric field. The gel 130 can be any one or more of a poly-acrylic acid copolymer, polyvinyl alcohol, a carboxylic acid copolymer or any other gelatinous or generally solid substance that can expel some form of solvent as the result of an applied electric field or current. In various embodiments, it can be useful for the gel 130 to contain ionized monomeric units, such as weak polyacids (e.g., a polyacrylic acid), strong polyacids (e.g., polysterene sulfonate), weak polybases (e.g., amine based) or strong polybases (“poly” here referring to polymerized units, thus part of a polymer gel network).

The solvent of gel 130 can be water, or any number of other solvents, e.g., an alcohol or some form of generally non-toxic substance, can be used depending on various circumstances, such as a medicating agent's solubility with the solvent. Various solvents may also contain micellar formulations enabling the solubilization of lipophilic compounds in a water-based formulation.

The medicating agent of gel 130 can be any number of drugs, pain-relievers, hormones (e.g., cortisone), stimulants or other substances that can be used for medically beneficial purposes and that may be absorbed through skin—human or otherwise—may be employed.

While one function of the gel 130 is to hold some form of solvent and medicating agent, a complementary function of the gel 130 is to controllably expel the solvent, which can act as a carrier for the medicating agent, upon command of the controller 114. This function can be accomplished by forming a voltage across (or a current through) the electrode pair via controller 114. FIGS. 2A and 2B depict a “before and after” example of this function of a solvent-bearing gel. As shown in FIG. 2A, the initial gel body 130 has a much larger volume than the solvent-depleted gel body 131 of FIG. 2B. The solvent in gel body 131 is depleted as a result of a reaction of the initial gel body 130 to electrical activity. The closing of switch 210, enables battery 112 to produce a voltage V₁ across the electrodes and to cause a current I₁ to pass through the initial gel body 130. The result of this electrical activity is that the gel body literally distorts and forces the solvent from its body, producing the solvent-depleted gel body 131. Details of such gels and their associated reactions can be found in: Osada Yoshihito & Gong Jian Ping. “Polymer gels and networks”, A. Khoklov & Y. Osada (eds.), pp. 177-217 (Marcel Dekker, New York Basel (2002) herein incorporated by reference.

Similar to FIGS. 2A and 2B, FIGS. 2C and 2D depict a process where a drug and solvent can be controllably expelled from a gel in response to the application of an electric field using a variant of the delivery system of FIG. 1A. As shown in FIG. 2C, the structure of the system 100 of FIG. 1A is changed such that the gel 130 is placed on the top of the substrate 110, and a cover/seal 240 is placed over the gel 130 to protect and seal the gel from the outside environment. A peel-able second seal 222 can be placed below the adhesive 220.

During operation (presumably after the removal of the second seal 222), the closing of switch 210 will enable the battery 112 to produce a voltage V₁ across the electrodes and cause current I₁ to pass through the gel 130. Again the result of this electrical activity is that the initial gel body 130 literally distorts and forces the solvent from its body. However, rather than pass directly to a skin surface, the solvent and medicating agent passes through a number of holes/pores 250 in the substrate 110 and then onto the skin surface below. The top cover/seal 240 can retain its shape during this process or optionally contract to stay in contact with the contracting gel body. An advantage of the illustrative embodiment of FIGS. 2C-2D is that the gel 130 does not need be in direct contact with the skin, thereby reducing risks of irritation caused by specific gel formulations (e.g. too basic or too acidic). Further, when relatively high DC voltages are applied (e.g. >2V) to the gel 130, water hydrolysis can occur near the electrodes rather quickly, thereby generating hydrogen or other undesired gases. By using the “reversed” geometry of FIGS. 2C-2D, the undesirable gases can more readily be diffused from the packaging.

For the illustrative embodiments of FIGS. 1-2D, sealing layers optionally can be added over the gel 130 to abate gel dehydration during storage as well as during use. Such sealing layers (not shown) may include thin polymer films (e.g., polyethylene or PET) coated with a diffusion barrier made of thin metal layers, such as aluminum or aluminum oxides, silicon oxides, silicon oxinitrides or multiple layers thereof.

While FIGS. 1-2D depict the use of a single electrode pair 140 having a circular shape, other electrode configurations can be used, including those shown in any of FIG. 3A (a pair of parallel electrodes A, B), FIG. 3B (a pair of parallel electrodes A, B with interleaved “teeth”) or FIG. 3C (a pair of curved, interleaved electrodes A, B). The electrode pairs A/B of FIGS. 3A-3C have common attributes in that they can be easily produced and can form generally even electric fields.

Additionally, it can be beneficial to use multiple pairs of electrodes. Various examples of multiple electrode pairs can be found in FIGS. 4A-4C.

FIG. 4A depicts a 3-by-3 electrode pair array 400 controllable by two sets of electrodes A, B, C and X, Y, Z. Because each circular electrode pair region 402 can be independently activated, a controller controlling the electrodes A, B, C and X, Y, Z can perform a greater variety of drug administration operations. For example, assuming that all nine of the electrode pair regions 402 are immersed in a gel having a uniform distribution of a common medicating agent, a controller controlling the various electrode pair regions 402 can separately and independently administer nine distinct doses of the medicating agent over preprogrammed intervals or in response to some external request.

Another advantage of using the array 400 of FIG. 4A is that a variety of different medicating agents can be independently administered. For example, if a first medicating agent covers the right three electrode pair regions 402 and a second medicating agent covers the left six electrode pair regions 402, a controller controlling the array 400 is free to administer either or both medicating agents at any given time. For various electrode configurations, such as shown in FIG. 4A, the use of an active matrix addressing scheme with an appropriate number of switches provides the greatest versatility of use with the least amount of hardware.

FIG. 4B depicts a variant of the multiple electrode pair concept where four electrodes are used to form three electrode pairs A-B, B-C and C-D with each of the three electrode pairs A-B, B-C and C-D being capable of administering a separate dose of one or more medical agents.

FIG. 4C shows yet another variant where three electrodes A, B and C form two electrode pairs A-B and B-C, which can be used to administer two separate doses of one or more medical agents.

For embodiments having a plurality of electrode pairs, it is advantageous to use a separate switch for each electrode in select embodiments, as opposed to the single switch in FIGS. 2A-2D. For example, while the array of FIG. 4C only needs two switches for electrodes A and C (electrode B being tied to ground), six separate switches (three to ground and three to a different voltage) are needed for the 3-by-3 array 400 of FIG. 4A in order to independently administer nine separate doses.

Further, in order to improve operational performance, each electrode A, B, C and X, Y, Z of array 400 may use multiple switches for different voltages—or use some other form of voltage/current control, such as a digital-to-analog converter, to vary the rates of drug administration.

Continuing to FIG. 5, a functional block diagram of a transdermal delivery system 500 is provided. Most of the elements 110-170 of FIG. 1A are shown, as well as an extra sensor 570 (buried in adhesive layer 120) and various internal components of the controller 114 including a timer 520, a switch array 510, a current sensor 512 (for sensing current passing through a particular pair of electrodes) and an analog-to-digital (“ADC”) converter 514 for monitoring sensors 170 and 570.

In operation and under power provided by the battery 112, the controller 114 is initialized via the user interface 160. In the present example, the user interface 116 is a combination of an activation button and a multicolored light-emitting diode with the activation button for initiating a drug administration or for starting a sequence of timed drug administrations, and the diode for indication system status, e.g., active/inactive/depleted, good/fail/fault etc. Optionally, the user interface 116 includes, or takes the form of, a computer-to-computer interface, such as a Firewire, USB or some specialized RFID-based system. In such instances, the transdermal system 500 can be both activated and programmed to apply certain medicating doses at precise intervals and/or for specific times.

Assuming that the transdermal system 500 is applied to a patient's skin with the controller 114 suitably programmed and activated, the controller 114 carries out its basic programming, which includes appropriately setting and resetting the timer 520, appropriately activating the electrode array 140 embedded in gel 130 to administer one or more medicating agents at proscribed times and monitoring the various sensors 512, 170 and 570 for feedback.

A first form of feedback is obtained from sensor 512, which monitors current passing through a pair of electrodes in the electrode array 140 embedded in the gel 130. The sensor 512 can be equally equipped to monitor voltages across a given electrode pair. By doing so, the controller 114 can effectively monitor the basic functionality of the electrode pair and/or monitor gel impedance, which can change as a function of how much solvent is present in the gel. Such information can be used to change basic operating parameters, such as the time duration for which a medicating dose will be administered or an intermittent time between doses. Such information may also be made available to the patient or attending medical staff via the user interface 116.

A second form of feedback is available through the second electrode array 160 located in the adhesive 120, where skin resistance is determined in order to provide biological information to the controller 114. Other possible forms of information obtained using electrode array 160 may include determining a resistance between a patient's skin and the gel 130, which can again provide useful operating parameters as well as some sort of indication that the transdermal system 500 is appropriately secure to the patient's skin.

In addition to use as a sensor, the second electrode array 160 also provides useful non-sensory functions. For example, by passing a current from the first electrode array 140, through the gel 130 and patient's skin and to the second electrode array 160, the transdermal system 500 takes advantage of iontophoresis (i.e., ElectroMotive Drug Administration (EMDA)) in order to better urge dermal penetration of drugs having certain molecular physical chemical parameters.

Other forms of feedback obtained through either or both of sensors 170 and 570 include determining whether transdermal system 500 is appropriately attached via skin resistance, monitoring skin temperature, monitoring heart rate (pulse rate) and/or blood oxygen (as a pulse-oximeter might), monitoring for skin irritation, swelling and so on, and providing basic self-testing functions, such as allowing the controller to determine whether a particular electrode is functional or a gel is depleted.

Also, either or both sensors 170 and 570 can be used for regulating administration of a particular drug. For example, assuming that an infrared pulse-oximeter is employed to measure pulse rate, various stimulants are deployed whenever a patient's pulse drops below a certain rate.

The above-identified embodiments have distinct advantages over any conventional drug delivery system. Highly portable and ergonomic drug-delivery systems can be precisely timed to deliver precise doses. Drugs having molecular structures subject to skin absorption can be administered in the form of a skin-patch. Further, the employment of an appropriate controller and user interface can allow a medical professional to monitor patient usage, e.g., monitor how many times a patient self-medicated and over what intervals. Finally, use of a controller allows for researchers to keep track of device performance during clinical tests as it could provide a detailed usage trace as well as act as a form of evidence.

The above-described systems and methods can be implemented using any of various known or later developed programming languages, such as “C”, “C++”, “FORTRAN”, Pascal”, “VHDL” and the like.

Accordingly, various storage media, such as magnetic computer disks, optical disks, electronic memories and the like, can contain information for directing a device, such as a computer, to implement the above-described systems and/or methods. Once an appropriate device has access to the information and programs contained on the storage media, the storage media can provide the information and programs to the device, thus enabling the device to perform the above-described systems and/or methods.

For example, a computer having a computer disk containing appropriate materials, such as a source file, an object file, an executable file or the like is configured and capable of performing the functions of the various systems and methods outlined in the diagrams and flowcharts above to implement the various functions. That is, the computer uses various portions of information from the disk relating to different elements of the above-described systems and/or methods, to implement the individual systems and/or methods and coordinate the functions of the individual systems and/or methods described above.

The various methods and devices described herein can be implemented in hardware and software. Further, the various methods and parameters are included by way of example only and not in any limiting sense. In view of this disclosure, those skilled in the art can implement the present teachings in determining their own techniques and needed equipment to effect these techniques, while remaining within the scope of the appended claims.

1. A transdermal drug delivery system (100) for providing controlled doses of a drug through the epidermis of a human or other animal, the transdermal drug delivery system (100) comprising:
a substrate (110) having an array of one or more electrode pairs (140) disposed thereon; and
a gel (130) disposed on the substrate (110) and in electrical contact with each electrode (142, 144) of the one or more electrode pairs (140), wherein the gel (130) contains at least a first medicating agent and is configured to change a rate of release of the first medicating agent based on at least one of a voltage or current provided by the electrode pairs (140).

2. The transdermal drug delivery system (100) of claim 1, further comprising a controller (114) coupled to each electrode (142, 144) of the one or more electrode pairs (140) configured to selectively provide a voltage across or current through each electrode pair (140).

3. The transdermal drug delivery system (100) of claim 2, further comprising a generally flat battery (112) disposed on the substrate (110).

4. The transdermal drug delivery system (100) of claim 3, further comprising a user interface (116) disposed on the substrate (110).

5. The transdermal drug delivery system (100) of claim 4, wherein the user interface (116) includes at least one or more of an activating device and a visual indicator.

6. The transdermal drug delivery system (100) of claim 5, wherein the user interface (116) includes at least an activating device, and wherein the controller (114) is configured to cause a change in the rate of release of the medicating agent as a response to the triggering of the activation device.

7. The transdermal drug delivery system (100) of claim 4, wherein the user interface (116) includes a communication port for enabling a computer to provide instructions to the controller (114) relating to the administration of the medicating agent.

8. The transdermal drug delivery system (100) of claim 2, further comprising a sensor (170, 512, 570) in communication with the controller (114) for providing information useful for the controlled administration of the first activating agent.

9. The transdermal drug delivery system (100) of claim 8, wherein the array of electrode pairs (140) includes two or more electrode pairs (140) with each electrode pair (140) capable of controlling drug release of a different portion of the gel (130).

10. The transdermal drug delivery system (100) of claim 9, wherein the gel (130) has at least two different medicating agents, and wherein the administration of each medicating agent is controllable by a different electrode pair (140).

11. The transdermal drug delivery system (100) of claim 2, wherein the controller (114) is configured to cause a change in the release of the medicating agent according to a predetermined time profile.

12. The transdermal drug delivery system (100) of claim 1, wherein the gel (130) is a polyelectrolyte gel (130) configured to expel a solvent in the presence of an electric field without reliance on iontophoresis.

13. The transdermal drug delivery system (100) of claim 1, wherein the gel (130) includes at least one of a poly-acrylic acid copolymer, polyvinyl alcohol and a carboxylic acid copolymer.

14. The transdermal drug delivery system (100) of claim 1, wherein the gel (130) consists of multiple layers of a pre-gel mixture cured using a polymerization process.

15. A transdermal drug delivery system (100) for providing controlled doses of a drug through the epidermis of a human or other animal, the transdermal drug delivery system (100) comprising:
a substrate (110) with a gel (130) disposed thereon, the gel (130) containing at least a first medicating agent;
a manipulating means (140) for actively manipulating a rate of release of the first medicating agent from the gel (130); and
a controlling means (114) for controlling the manipulating means.

16. The transdermal drug delivery system (100) of claim 15, wherein the manipulating means (140) includes at least one pair of electrodes (142, 144) capable of providing an electric field to the gel (130), and wherein the gel (130) is configured to change the rate in which it expels a solvent in response to electric fields.

17. The transdermal drug delivery system (100) of claim 15, wherein the controlling means (114) includes a microcontroller (114) having a timer (520).

18. The transdermal drug delivery system (100) of claim 17, further comprising a visual indicator (116) coupled to the microcontroller (114) for conveying information to a human about the status of the transdermal drug delivery system (100).

19. A transdermal drug delivery system (100) for providing controlled doses of a drug through the epidermis of a human or other animal, the transdermal drug delivery system (100) comprising:
a substrate (110) having a first array of one or more first electrodes (140) and a second array of one or more second electrodes (160) disposed thereon;
a polyelectrolyte gel (130) disposed on the substrate (110) and in electrical contact with each first electrode (140);
a controller (114) disposed on the substrate (110) and capable of controlling an electric current to pass from the one or more first electrodes (140) and through the gel (130) and a patient's skin to the one or more second electrodes (160) such that the electric current causes the gel (130) to actively release a medicating agent contained in the gel (130) onto the patient's skin using a non-iontophoresis process and further coax the medicating agent to penetrate the patient's skin using iontophoresis.

20. The transdermal drug delivery system (100) of claim 19, wherein the controller (114) is also capable of causing an appreciable amount of electric current to pass between two first electrodes (142, 144).
